(19) United States
(12) Patent Application Publication
(10) Pub. No.: US 2017/0113250 A1
(43) Pub. Date: Apr. 27, 2017

(54) ULTRASOUND PROBE

(71) Applicant: ALPINION MEDICAL SYSTEMS CO., LTD., Hwaseong-si (KR)

(72) Inventors: Wonseok Lee, Daegu (KR); Susung Lee, Yongin-si (KR); Eunhee Shin, Seoul (KR)

(21) Appl. No.: 15/335,820

(22) Filed: Oct. 27, 2016

(30) Foreign Application Priority Data
Oct. 27, 2015 (KR) 10-2015-0149757

(51) Int. Cl. B06B 1/06; A61B 8/00

(52) U.S. Cl. CPC B06B 1/06; A61B 8/4444; A61B 8/4494

(57) ABSTRACT

Provided is an ultrasound probe and more particularly, to an ultrasound probe capable of improving the quality of an ultrasound image. The ultrasound probe includes a piezoelectric element unit that transduces an electrical signal and an ultrasound signal in to each other, and a matching layer that is located in front of the piezoelectric element unit to reduce a difference in sound impedance between the piezoelectric element unit and a target object. The piezoelectric element unit may include multiple piezoelectric layers laminated in a forward and backward direction, and each of the multiple piezoelectric layers may have a non-uniform cross-section structure in which a thickness varies in an elevation direction orthogonal to the forward and backward direction.

### TECHNICAL FIELD

The present disclosure relates to an ultrasound probe and more particularly, to an ultrasound probe capable of improving the quality of an ultrasound image.

### BACKGROUND

Along with the social change in which the importance of human health and happiness has been increasing, the importance of medical devices has been increased. In the medical device-related industry, ultrasound image diagnostic devices accounts for a greater and greater portion. The most important function of a ultrasound image diagnostic device is an image quality, and one of the most important factors determining an image quality is an ultrasound transducer. Therefore, a high-performance ultrasound transducer is essential for a high-definition ultrasound image diagnostic device.

The most representative ultrasound device used for medical purposes may be an ultrasound image diagnostic device mainly used for imaging organs within a human body and a fetus. Unlike other medical imaging devices for imaging the inside of a human body such as an X-ray imaging machine, a computed tomography device (CT), or a magnetic resonance imaging device (MRI), the ultrasound image diagnostic device enables a diagnostician to image a desired specific site within a human body by discretionally steering an ultrasound-radiation angle and does not give damage, such as radiation damage, to the human body. Also, the ultrasound image diagnostic device can acquire an image in a shorter time than the other medical imaging devices for imaging the inside of a human body. In order to implement an image with the ultrasound image diagnostic device, a means and/or a device for transducing an ultrasound signal and an electrical signal into each other is needed, and in the art, this is called an ultrasound probe or an ultrasound transducer.

FIG. 1 is a diagram illustrating a configuration of a conventional medical ultrasound transducer. As illustrated in FIG. 1, a conventional medical ultrasound transducer 10 includes a piezoelectric layer 11 in which a piezoelectric material vibrates to transduce an electrical signal and a sound signal into each other, a matching layer 12 that reduces a difference in sound impedance between the piezoelectric layer 11 and a human body, a lens layer 13 that focuses an ultrasound propagating to the front of the piezoelectric layer 11, and a backing layer 14 that blocks propagation of an ultrasound to the back of the piezoelectric layer 11 and thus suppresses image distortion. In the conventional ultrasound transducer 10, the piezoelectric layer 11 has a single structure, so that there is a limitation in efficient focusing of an ultrasound beam, which may cause deterioration in resolution of an ultrasound image and penetration function. As a result, the conventional medical ultrasound transducer may have a limitation in acquiring precise image information.

Meanwhile, in most of conventional ultrasound probes adopting a multilayered PZT structure among various conventional ultrasound probes, mode coupling occurs at a specific aspect ratio due to structural reasons, which has exerted a negative influence on probe design and acquisition of a high-definition image. To be specific, in a conventional ultrasound probe having a multilayered PZT structure, an active element is diced to separate a channel of the active element. Such a process causes deterioration in the aspect ratio of the active element, so that vibrations in directions (i.e., elevation direction and azimuth direction) different from a depth direction of the active element cause mode coupling that affects the depth direction. Therefore, the above-described prior art has a limitation in improving sound characteristics of the probe and acquiring a high-definition ultrasound image.

The background technology of the present disclosure is disclosed in Koran Patent Laid-open Publication No. 10-2013-0097550.

### SUMMARY

In view of the foregoing, the present disclosure provides an ultrasound probe capable of improving the quality of an ultrasound image.

Further, the present disclosure improves sound characteristics of a probe by applying a structure which is un-uniform in an elevation direction to a conventional multilayered active layer structure which exhibits good electrical matching between an image system and a probe and thus improving an aspect ratio.

Furthermore, the present disclosure provides a probe having an un-uniform multilayered active layer structure.

Moreover, the present disclosure reduces crosstalk caused by mode coupling of an active layer (which means a piezoelectric layer) to improve an aspect ratio of each active layer and thus increases a sensitivity and a bandwidth and reduces a ring-down time (i.e., a time required for a sound wave to decrease to a specific size on the basis of a main bang in a waveform).

Further, the present disclosure improves the quality of an ultrasound image through using an apodization effect in an elevation direction by improving a beam profile in the elevation direction.

Furthermore, the present disclosure improves the quality of an ultrasound image, particularly a near-field image, by making an elevation direction-beamwidth uniform along a depth.

Moreover, the present disclosure improves electrical matching with respect to an image system by reducing impedance.

However, problems to be solved by the present disclosure are not limited to the above-described problems. There may be other problems to be solved by the present disclosure.

According to an aspect of the present disclosure, there is provided an ultrasound probe including a piezoelectric element unit that transduces an electrical signal and an ultrasound signal in to each other, and a matching layer that is located in front of the piezoelectric element unit to reduce a difference in sound impedance between the piezoelectric element unit and a target object. The piezoelectric element unit may include multiple piezoelectric layers laminated in a forward and backward direction, and each of the multiple piezoelectric layers may have a non-uniform cross-section structure in which a thickness varies in an elevation direction orthogonal to the forward and backward direction.

Herein, the piezoelectric element unit may include a first piezoelectric layer and a second piezoelectric layer located in front of the first piezoelectric layer, and any one of the first piezoelectric layer and the second piezoelectric layer may include a middle portion thicker than side portions in the elevation direction, and the other one of the first piezoelectric layer and the second piezoelectric layer may include a middle portion thinner than side portions in the elevation direction.

Further, in each of the first piezoelectric layer and the second piezoelectric layer, a middle portion and a side portion may be provided with a space therebetween in the elevation direction. A flexible printed circuit board (FPCB) may be interposed between the first piezoelectric layer and the second piezoelectric layer, and the space may correspond to a thickness of the flexible printed circuit board. Further, any one of the first piezoelectric layer and the second piezoelectric layer may be the second piezoelectric layer, and the other one of the first piezoelectric layer and the second piezoelectric layer may be the first piezoelectric layer.

Furthermore, a front surface of a middle portion in the first piezoelectric layer may be recessed to the back of front surfaces of side portions and thus face a back surface of a middle portion in the second piezoelectric layer, and the back surface of the middle portion in the second piezoelectric layer may be protruded to the back of back surfaces of side portions. Moreover, the first piezoelectric layer and the second piezoelectric layer are protruded and recessed to be engaged with each other with a flexible printed circuit board (FPCB) interposed therebetween. Further, a back surface of the first piezoelectric layer and a front surface of the second piezoelectric layer may be formed flat.

Further, the middle portion of the first piezoelectric layer may be recessed in a stepwise manner with respect to the side portions, and the middle portion of the second piezoelectric layer may be protruded in a stepwise manner with respect to the side portions.

Further, the ultrasound probe of the present disclosure may further include a backing layer that is located in back of the piezoelectric element unit to block an ultrasound signal propagating to the back of the piezoelectric element unit, and a lens layer that is located in front of the matching layer to focus an ultrasound signal propagating to the front of the piezoelectric element unit on the target object.

The above-described exemplary embodiments are provided by way of illustration only and should not be construed as liming the present disclosure. Besides the above-described exemplary embodiments, there may be additional exemplary embodiments described in the accompanying drawings and the detailed description.

The present disclosure has an effect of providing an ultrasound probe capable of improving the quality of an ultrasound image.

The present disclosure has an effect of improving sound characteristics of a probe by applying a structure which is un-uniform in an elevation direction to a conventional multilayered active layer structure which exhibits good electrical matching between an image system and a probe and thus improving an aspect ratio.

The present disclosure has an effect of reducing crosstalk caused by mode coupling of an active layer to improve an aspect ratio of each active layer and thus increasing a sensitivity and a bandwidth and reducing a ring-down time (i.e., a time required for a sound wave to decrease to a specific size on the basis of a main bang in a waveform).

The present disclosure improves the quality of an ultrasound image through using an apodization effect in elevation direction by improving a beam profile in the elevation direction.

The present disclosure has an effect of improving the quality of an ultrasound image, particularly a near-field image, by making an elevation direction-beamwidth uniform along a depth.

The present disclosure has an effect of improving electrical matching with respect to an image system by reducing impedance.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 is a diagram illustrating a configuration of a conventional medical ultrasound transducer;

FIG. 2 is a schematic configuration view of an ultrasound diagnostic device including a probe according to an exemplary embodiment of the present disclosure;

FIG. 3 is a perspective view of an ultrasound probe according to an exemplary embodiment of the present disclosure;

FIG. 4 is a diagram three-dimensionally illustrating a partially cut transducer within a probe according to an exemplary embodiment of the present disclosure;

FIG. 5 is a cross-sectional view of a transducer within a probe according to an exemplary embodiment of the present disclosure;

FIG. 6 is a cross-sectional view of a piezoelectric layer according to an exemplary embodiment of the present disclosure;

FIG. 7 is a graph showing the result of a first experiment according to an exemplary embodiment of the present disclosure;

FIG. 8 is a graph showing the result of a second experiment according to an exemplary embodiment of the present disclosure;

FIG. 9 is a graph showing the result of a third experiment according to an exemplary embodiment of the present disclosure; and

FIG. 10 is a graph showing the result of a fourth experiment according to an exemplary embodiment of the present disclosure.

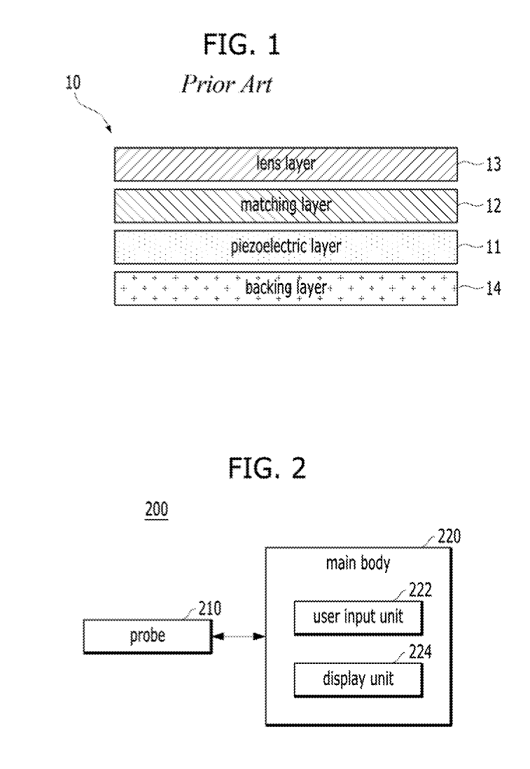

### DETAILED DESCRIPTION

Hereinafter, exemplary embodiments of the present disclosure will be described in detail with reference to the accompanying drawings. If it is considered that description of related known configuration or function may cloud the gist of the present disclosure, the description thereof will be omitted. Further, specific values described in the exemplary embodiments are just examples.

The present disclosure relates to an ultrasound probe and particularly to a structure of a transducer included in an ultrasound probe. The present disclosure can improve sound characteristics of a probe by applying a structure which is un-uniform in an elevation direction to a conventional multilayered active layer structure thus improve an aspect ratio of an active layer, and on the basis of this improvement, the present disclosure can improve a beam profile in the elevation direction and thus improve the quality of an ultrasound image.

FIG. 2 is a schematic configuration view of an ultrasound diagnostic device including a probe according to an exemplary embodiment of the present disclosure.

Referring to FIG. 2, an ultrasound diagnostic device 200 according to exemplary embodiment of the present disclosure may roughly include a probe 210 and a main body 220. The main body 220 may include a user input unit 222 and a display unit 224. Although the ultrasound diagnostic device 200 of the present disclosure is described as including only the probe 210, the main body 220, the user input unit 222, and the display unit 224 in the present exemplary embodiment, this is provided only for illustration of the technical concept of the present disclosure, and it would be understood by those skilled in the art that various changes and modifications may be made to the components included in the ultrasound diagnostic device 200 without changing essential features of the present disclosure.

To put it briefly, the ultrasound diagnostic device 200 includes the probe 210 configured to radiate an ultrasound signal to an inspection target object (target object) and receive an ultrasound echo signal from the inspection target object and the main body 220 equipped with the user input unit 222 and the display unit 224 and configured to generate an image of the inspection target object.

The ultrasound diagnostic device 200 operates to receive an instruction made by user manipulation or input through the user input unit 222 and radiate an ultrasound signal to the inspection target object and receive an ultrasound echo signal reflected from the inspection target object through the probe 210 to generate a received signal, and also operates to generate an ultrasound image on the basis of the received signal through the main body 220 and output the generated image through the display unit 224. Herein, the probe 210 is in direct contact with a diagnostic site of the inspection target object, and may access the main body 220 through a cable or connector connected to the main body 220 as one body.

Further, the probe 210 may include a beamformer (not illustrated) that operates to transmit and receive ultrasound signals and perform transmit-focusing and receive-focusing of ultrasounds. Herein, the probe 210 may include multiple 1D (dimension) or 2D array transducers, and the probe 210 transmits an ultrasound beam, which is focused by appropriately delaying an input time of a pulse inputted to each transducer, to the inspection target object along a transmission scanline. Meanwhile, the ultrasound echo signal reflected from the inspection target object is inputted to each transducer with a different reception time, and each transducer outputs the inputted ultrasound echo signal to the beamformer. The beamformer focuses an ultrasound signal on a specific site by adjusting driving timing of each transducer within the probe 210 when the probe 210 transmits the ultrasound signal, and focuses an ultrasound echo signal by adding a time delay to each ultrasound echo signal of the probe 210, considering the difference in time required for an ultrasound echo signal reflected from the inspection target object to reach each transducer of the probe 210.

The main body 220 operates to generate an ultrasound image on the basis of the received signal generated using the ultrasound echo signal received through the probe 210 and output the image through the display unit 224.

The user input unit 222 receives an instruction made by user manipulation or input. Herein, the user instruction may be a setup instruction for controlling the ultrasound diagnostic device 200. Hereinafter, a structure of the probe 210 suggested in the present disclosure will be described in more detail.

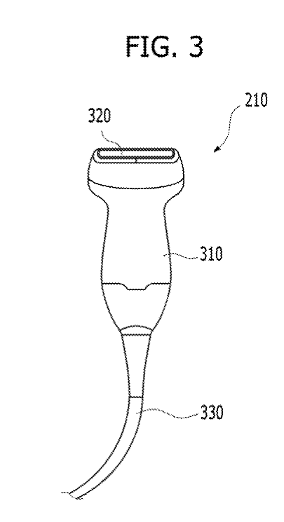

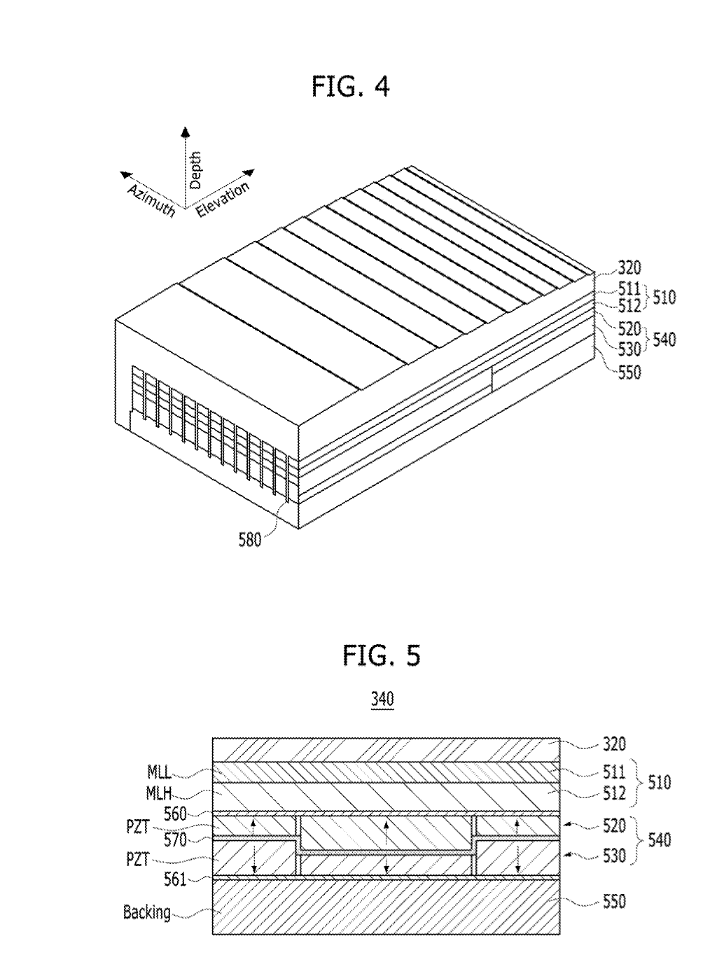

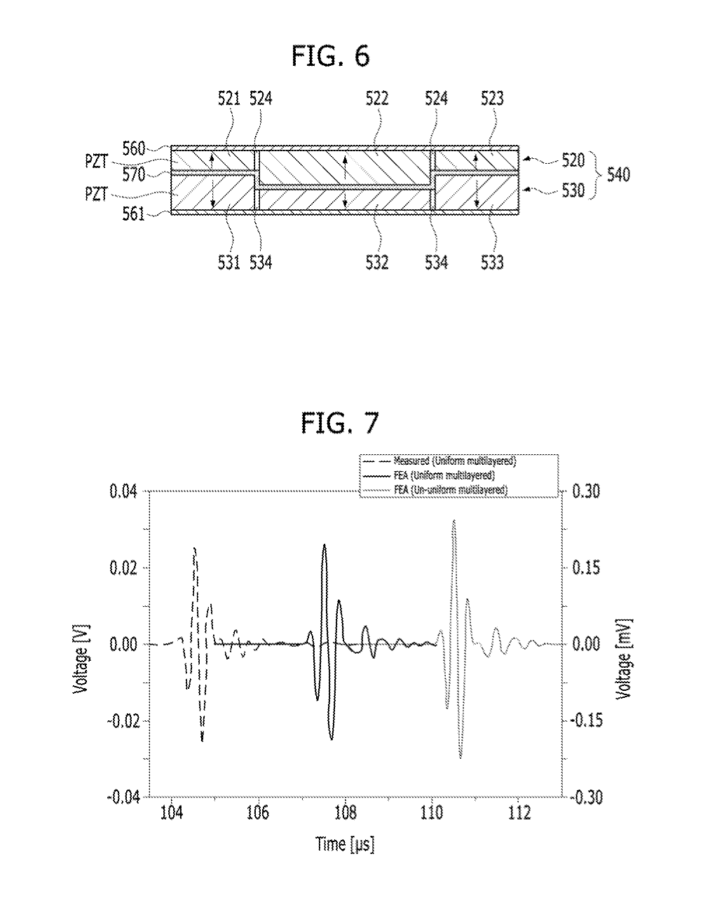

FIG. 3 is a perspective view of an ultrasound probe according to an exemplary embodiment of the present disclosure, FIG. 4 is a diagram three-dimensionally illustrating a partially cut transducer within a probe according to another exemplary embodiment of the present disclosure, FIG. 5 is a cross-sectional view of a transducer within a probe according to an exemplary embodiment of the present disclosure, and FIG. 6 is a cross-sectional view of a piezoelectric layer according to an exemplary embodiment of the present disclosure.

Referring to FIG. 3 through FIG. 6, the ultrasound probe 210 according to an exemplary embodiment of the present disclosure may include a housing 310, a lens 320, and a cable 330.

The housing 310 is a cover that covers an internal module of the probe 210 and constitutes a body of the probe 210. The housing 310 may include a transducer 340 configured to radiate an ultrasound signal, receive an ultrasound echo signal, and transduce the received ultrasound echo signal. In the transducer 340 included in the housing 310, an ultrasound signal may be generated depending on whether or not a voltage is applied from the ultrasound diagnostic device 200. Details of the transducer 340 will be described with reference to FIG. 4 through FIG. 6.

The lens 320 radiates an ultrasound signal, receives an ultrasound echo signal, and is in contact with a diagnostic site such as the skin of the inspection target object. Further, the lens 320 enables an ultrasound signal to be focused on a position of the inspection target object and receives an ultrasound echo signal reflected from the inspection target object. The cable 330 connects the main body 220 of the ultrasound diagnostic device 200 and the housing 310 of the probe 210, and the cable 330 may transmit the ultrasound echo signal to the main body 220.

Hereinafter, the detailed structure of the transducer 340 included in the probe 210 will be described with reference to FIG. 4 through FIG. 6. Herein, FIG. 5 and FIG. 6 illustrate schematic cross-sectional views of the transducer illustrated in FIG. 4 cut along an elevation direction. That is, in the cross-sectional views illustrated in FIG. 5 and FIG. 6, an azimuth direction is a normal line.

Referring to FIG. 4 through FIG. 6, the transducer 340 according to an exemplary embodiment of the present disclosure may roughly include the lens layer 320, a matching layer 510, a piezoelectric layer 540, and a backing layer 550.

The transducer 340 may have a structure in which the backing layer 540, the piezoelectric layer 540, the matching layer 510, and the lens layer 320 are laminated in sequence in a direction in which an ultrasound signal is radiated from the probe 210 to the inspection target object (target object) (i.e., forward or depth direction).

In the piezoelectric layer 540, a piezoelectric element (or piezoelectric material, piezoelectric body) vibrates to transduce an electrical signal and a sound signal into each other.

The piezoelectric layer 540 may be formed by laminating multiple piezoelectric layers along a forward and backward direction. The multiple piezoelectric layers may include a first piezoelectric layer 530 and a second piezoelectric layer 520. Herein, the second piezoelectric layer 520 may be located in front and the first piezoelectric layer 530 may be located at the back. However, the multiple piezoelectric layers are not necessarily limited to two layers, and may include three or more piezoelectric layers.

The piezoelectric layer 540 is formed of a piezoelectric element, and in the present disclosure, PZT may be used as the piezoelectric element. That is, in each of the first piezoelectric layer 530 and the second piezoelectric layer 520, PZT may be used as a piezoelectric element or an active element, but the present disclosure is not limited thereto. For example, in the ultrasound probe, piezoelectric ceramic, piezo-composite, or piezoelectric single crystal may be used as the piezoelectric element. Herein, referring to FIG. 4, the piezoelectric layer 540 may be partitioned by kerfs 580 formed with a space therebetween along the azimuth direction.

PZT is a material of a solid solution of lead zirconate titanate, and it is easy to prepare and exhibits high piezoelectric and dielectric properties. In a microscopic view of the PZT having a perovskite structure, the PZT includes a grain boundary and a dipole within the grain boundary. The dipole has polarity. However, each dipole faces in a different direction, and, thus, the overall polarity is 0. In this case, there is no piezoelectric property. However, piezoelectric properties are generated through a poling process of applying a voltage to a piezoelectric body. That is, if electrical energy is applied to the piezoelectric body, the dipoles undergo polarization in which all the dipoles are aligned in one direction through polarization rotation, and, thus, the piezoelectric body can be driven.

The second piezoelectric layer 520 and the first piezoelectric layer 530 may be respectively laminated in front and back of a flexible printed circuit board (FPCB) 570. That is, the second piezoelectric layer 520, the FPCB 570, and the first piezoelectric layer 530 may be laminated in sequence in a direction in which an ultrasound signal is radiated to the inspection target object (target object) (i.e., forward or depth direction).

Further, each of the second piezoelectric layer 520 and the first piezoelectric layer 530 may have a non-uniform cross-section structure in which a thickness varies in the elevation direction orthogonal to the forward and backward direction.

In most of conventional ultrasound probe technologies adopting a multilayered PZT structure, mode coupling occurs at a specific aspect ratio due to structural reasons, which has exerted a negative influence on probe design and acquisition of a high-definition image. For reference, the mode coupling refers to a phenomenon in which vibrations in directions (i.e., elevation direction and azimuth direction) different from a depth direction of an active element affect the depth direction.

According to the present disclosure, in order to solve the problem occurring at a specific aspect ratio, each of the second piezoelectric layer 520 and the first piezoelectric layer 530 has a non-uniform cross-section structure in which a thickness varies in the elevation direction. That is, each of the second piezoelectric layer 520 and the first piezoelectric layer 530 is formed to have a structure having an un-uniform thickness in the elevation direction. Further, according to the present disclosure, in order to avoid a specific aspect ratio, each of the second piezoelectric layer 520 and the first piezoelectric layer 530 may include multiple piezoelectric elements, and the multiple piezoelectric elements may be electrically isolated from each other.

That is, the first piezoelectric layer 530 may include multiple piezoelectric elements 531, 532, and 533 and the second piezoelectric layer 520 may also include multiple piezoelectric elements 521, 522, and 523. Herein, the second piezoelectric layer 520 may include a middle portion 522 thicker than side portions 521 and 523 in the elevation direction, and the first piezoelectric layer 530 may include a middle portion 532 thinner than side portions 531 and 533 in the elevation direction. Further, in order to improve an aspect ratio, the middle portion 522 and the side portions 521 and 523 of the second piezoelectric layer 520 may be provided with a second space 524 therebetween in the elevation direction, and the middle portion 532 and the side portions 531 and 533 of the first piezoelectric layer 530 may be provided with a first space 534 therebetween in the elevation direction.

Further, a front surface of the middle portion 532 in the first piezoelectric layer 530 may be recessed more backward than front surfaces of the side portions 531 and 533 so as to face a back surface of the middle portion 522 in the second piezoelectric layer 520, and the back surface of the middle portion 522 in the second piezoelectric layer 520 may be protruded more backward than back surfaces of the side portions 521 and 523. Furthermore, the middle portion 532 of the first piezoelectric layer 530 may be recessed in a stepwise manner with respect to the side portions 531 and 533, and the middle portion 522 of the second piezoelectric layer 520 may be protruded in a stepwise manner with respect to the side portions 521 and 523.

Moreover, the first piezoelectric layer 530 and the second piezoelectric layer 520 may be protruded and recessed to be engaged with each other with the flexible printed circuit board (FPCB) 570 interposed therebetween.

Referring to FIG. 5 and FIG. 6, the middle portion 522 of the second piezoelectric layer 520 may be provided as being protruded to be fitted and engaged with a recessed shape (width in the elevation direction or thickness in the depth direction) of the middle portion 532 in the first piezoelectric layer 530. Further, the side portions 521 and 523 of the second piezoelectric layer 520 may be provided to be relatively recessed with respect to the middle portion 522 of the second piezoelectric layer 520 to be fitted and engaged with the side portions 531 and 533 relatively protruded with respect to the middle portion 532 of the first piezoelectric layer 530. Herein, such a shape to be fitted and engaged may be set considering the thickness and the degree of bending of the flexible printed circuit board 570 interposed between the first piezoelectric layer 530 and the second piezoelectric layer 520.

Further, a back surface of the first piezoelectric layer 530 and a front surface of the second piezoelectric layer 520 may be formed flat. Herein, referring to FIG. 5 and FIG. 6, being formed flat may mean that in the first piezoelectric layer 530 or the second piezoelectric layer 520, side portions are formed to maintain the same level (depth) without being relatively protruded or recessed with respect to a middle portion.

Meanwhile, the first piezoelectric layer 530 or the second piezoelectric layer 520 formed of PZT generates a lower frequency as a thickness thereof increases. When the first piezoelectric layer 530 or the second piezoelectric layer 520 has a lower frequency such as a low frequency, they are subject to less attenuation. Therefore, it may be more advantageous for forward propagation of an ultrasound signal to avoid a specific aspect ratio in a direction in which a thickness of the second piezoelectric layer 520 located in front is increased (e.g., a thickness of the middle portion 522 is increased). Further, according to the present disclosure, the middle portion 522 of the second piezoelectric layer 520 is formed thicker than the side portions 521 and 523, so that a high sound pressure is generated in the middle portion 522. Thus, an ultrasound can be more uniformly transferred in the depth direction (i.e., direction in which the ultrasound signal is irradiated to the inspection target object (target object), or forward direction) due to an apodization effect. Therefore, the quality of an ultrasound image can be improved.

In an exemplary embodiment of the present disclosure, it has been described that the middle portion 532 of the first piezoelectric layer 530 is recessed in a stepwise manner with respect to the side portions 531 and 533, and the middle portion 522 of the second piezoelectric layer 520 is protruded in a stepwise manner with respect to the side portions 521 and 523, but the present disclosure is not limited thereto. On the contrary, for example, the middle portion 532 of the first piezoelectric layer 530 may be protruded in a stepwise manner with respect to the side portions 531 and 533, and the middle portion 522 of the second piezoelectric layer 520 may be recessed in a stepwise manner with respect to the side portions 521 and 523.

Meanwhile, the flexible printed circuit board (FPCB) 570 may be interposed between the first piezoelectric layer 530 and the second piezoelectric layer 520, and the second space 524 between the middle portion 522 and the side portions 521 and 523 of the second piezoelectric layer 520 and the first space 534 between the middle portion 532 and the side portions 531 and 533 of the first piezoelectric layer 530 may be formed corresponding to a thickness of the flexible printed circuit board 570. Since the piezoelectric layers are partitioned to be electrically isolated from each other by setting the first space 534 and the second space 524, the present disclosure has an effect of improving an aspect ratio of the piezoelectric layer 540.

The multiple piezoelectric elements 531, 532, and 533 of the first piezoelectric layer 530 and the multiple piezoelectric elements 521, 522, and 523 of the second piezoelectric layer 520 may be respectively laminated in front and back of the flexible printed circuit board 570. Thus, the flexible printed circuit board 570 may electrically connect the multiple piezoelectric elements 531, 532, and 533 included in the first piezoelectric layer 530 to the multiple piezoelectric elements 521, 522, and 523 included in the second piezoelectric layer 520, respectively.

The flexible printed circuit board 570 can be precision-processed and can be uniformly manufactured with ease. Further, in the present disclosure, the flexible printed circuit board 570 is used as a signal line, and a second GRound Sheet (GRS) 560 may be interposed between the second piezoelectric layer 520 and the matching layer 510 and a first GRound Sheet (GRS) 561 may be interposed between the first piezoelectric layer 530 and the backing layer 550.

Further, in the present disclosure, the first piezoelectric layer 520 and the first piezoelectric layer 530 are formed to have un-uniform thicknesses in the elevation direction, and, thus, an aspect ratio of the piezoelectric layer 540 can be improved to improve sound characteristics of the probe. Therefore, on the basis of this improvement, the present disclosure can improve a beam profile in the elevation direction and thus improve the quality of an ultrasound image.

The matching layer 510 is located in front of the piezoelectric layer 540 and reduces a difference in sound impedance between the piezoelectric layer 540 and the target object in order for an ultrasound signal generated in the piezoelectric layer 540 to be transferred as much as possible to a specific site of the target object.

The matching layer 510 may include a Matching Layer High (MLH) 512 and a Matching Layer Low (MLL) 511, and the MLL 511 may be located in front of the MLH 512 and the MLH 512 may be located in front of the second GRS 560.

The matching layer 510 is provided to transmit and receive an ultrasound generated in the piezoelectric layer 540 to the inside of the target object with high efficiency, and functions to match a sound impedance of the piezoelectric layer 540 stage by stage to be close to a sound impedance of the inspection target object.

The lens layer 320 is located in front of the matching layer 510 and focuses an ultrasound signal propagating to the front of the piezoelectric layer 540 on a specific site of the target object.

For example, the lens layer 320 may be configured as a sound lens formed of silicone rubber having sound impedance dose to that of a living body. Herein, the sound lens constituting the lens layer 320 may have various shapes, such as a center-convex shape and a flat shape, depending on the designers' design.

The backing layer 550 is located in back of the piezoelectric layer 540 and blocks propagation of an ultrasound to the back of the piezoelectric layer 540 and thus suppresses image distortion.

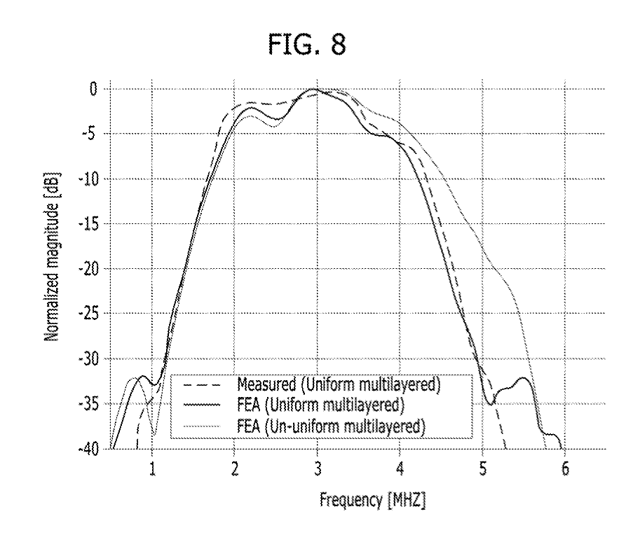

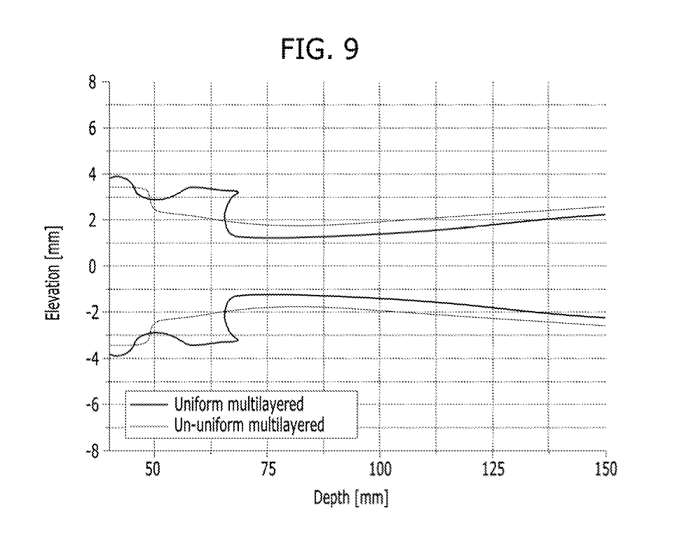

Meanwhile, FIG. 7 and FIG. 8 illustrate the results of experiments comparing the performance of a conventional ultrasound probe and the performance of an ultrasound probe according to an exemplary embodiment of the present disclosure.

FIG. 7 is a graph showing the result of a first experiment according to an exemplary embodiment of the present disclosure, and FIG. 8 is a graph showing the result of a second experiment according to an exemplary embodiment of the present disclosure. FIG. 7 shows a waveform graph, and FIG. 8 shows a frequency spectrum graph.

In FIG. 7 and FIG. 8, “Measured (Uniform multilayered)” represents a graph measured using the conventional ultrasound probe including layers with a uniform thickness (i.e., ultrasound probe to which a conventional multilayered active layer structure is applied), “FEA (Uniform multilayered)” represents a graph obtained by applying FEA (Finite-Element Analysis) to values of “Measured (Uniform multilayered)”, and “FEA (Un-uniform multilayered)” represents a graph obtained by applying FEA to values measured using the ultrasound probe of the present disclosure (i.e., ultrasound probe to which an un-uniform multilayered active layer structure is applied).

In this regard, the measured FEA (Uniform multilayered) values and FEA (Un-uniform multilayered) values for fractional bandwidth, sensitivity, and ring-down time are as follows.

In the FEA (Uniform multilayered) graph, a fractional bandwidth at −6 dB has a value of 74.8%, a sensitivity has a value of −68.6 dB, and a ring-down time at −20 dB has a value of 1.14 μs, and in the FEA (Un-uniform multilayered) graph, a fractional bandwidth at −6 dB has a value of 75.9%, a sensitivity has a value of −66.3 dB, and a ring-down time at −20 dB has a value of 1.12 μs.

According to these graphs, the ultrasound probe adopting the conventional technology has a sensitivity value of −68.6 dB and a fractional bandwidth value of 74.8%, whereas the ultrasound probe adopting the technology of the present disclosure has a sensitivity value of −66.3 dB and a fractional bandwidth value of 75.9%. Therefore, it can be seen that the present disclosure increases a sensitivity and a bandwidth as compared with the conventional technology. Further a ring-down time value is 1.14 μs in case of adopting the conventional technology and 1.02 μs in case of adopting the present disclosure. Therefore, it can be seen that the present disclosure reduces a ring-down time as compared with the conventional technology.

As such, it can be seen that the ultrasound probe adopting the technology of the present disclosure reduces crosstalk caused by mode coupling of an active layer (which means a piezoelectric layer) to improve an aspect ratio of each active layer and thus increases a sensitivity and a bandwidth and reduces a ring-down time.

Meanwhile, FIG. 9 is a graph showing the result of a third experiment according to an exemplary embodiment of the present disclosure. FIG. 9 shows a beamwidth graph measured using the conventional ultrasound probe (i.e., ultrasound probe to which a conventional multilayered active layer structure is applied) and the ultrasound probe of the present disclosure (i.e., ultrasound probe to which an un-uniform multilayered active layer structure is applied).

On the basis of FIG. 9, the present disclosure adopts an un-uniform multilayered active layer structure to the ultrasound probe, and thus can improve the quality of an ultrasound image, particularly a near-field image, by making an elevation direction-beamwidth uniform along a depth.

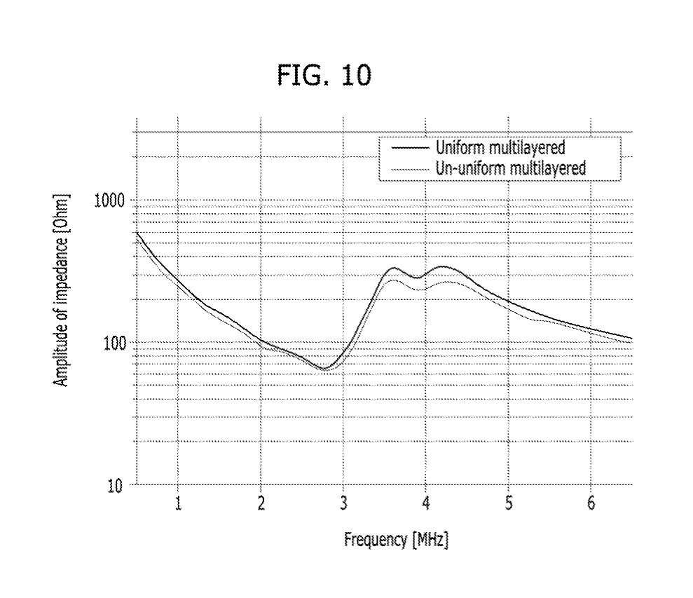

Further, FIG. 10 is a graph showing the result of a fourth experiment according to an exemplary embodiment of the present disclosure. FIG. 10 shows an impedance amplitude graph measured using the conventional ultrasound probe (i.e., ultrasound probe to which a conventional multilayered active layer structure is applied) and the ultrasound probe of the present disclosure (i.e., ultrasound probe to which an un-uniform multilayered active layer structure is applied).

On the basis of FIG. 10, the ultrasound probe of the present disclosure can reduce impedance equivalently or more efficiently than the conventional ultrasound probe having a multilayered active layer structure including layers with a uniform thickness in an elevation direction. Further, the present disclosure can improve electrical matching with respect to an image system by reducing impedance.

The present disclosure has been described by specified matters such as specific components and limited exemplary embodiments and drawings in the exemplary embodiment of the present disclosure as described above, this is just provided to assist more overall appreciation and the present disclosure is not limited to the exemplary embodiment. Various modifications and changes can be made by those skilled in the art from the descriptions above.

Accordingly, the spirit of the present disclosure is defined by the appended claims rather than by the description preceding them, and it should be appreciated that all technical spirit which are evenly or equivalently modified are included in the claims of the present disclosure.

We claim:

1. An ultrasound probe comprising:
a piezoelectric element unit that transduces an electrical signal and an ultrasound signal in to each other; and
a matching layer that is located in front of the piezoelectric element unit to reduce a difference in sound impedance between the piezoelectric element unit and a target object,
wherein the piezoelectric element unit includes multiple piezoelectric layers laminated in a forward and backward direction,
each of the multiple piezoelectric layers has a non-uniform cross-section structure in which a thickness varies in an elevation direction orthogonal to the forward and backward direction,
the piezoelectric element unit includes a first piezoelectric layer and a second piezoelectric layer located in front of the first piezoelectric layer,
any one of the first piezoelectric layer and the second piezoelectric layer includes a middle portion thicker than side portions in the elevation direction, and
the other one of the first piezoelectric layer and the second piezoelectric layer includes a middle portion thinner than side portions in the elevation direction.

2. The ultrasound probe of claim 1,
wherein in each of the first piezoelectric layer and the second piezoelectric layer, a middle portion and a side portion are provided with a space therebetween in the elevation direction.

3. The ultrasound probe of claim 2,
wherein a flexible printed circuit board (FPCB) is interposed between the first piezoelectric layer and the second piezoelectric layer, and
the space corresponds to a thickness of the flexible printed circuit board.

4. The ultrasound probe of claim 1,
wherein any one of the first piezoelectric layer and the second piezoelectric layer is the second piezoelectric layer, and
the other one of the first piezoelectric layer and the second piezoelectric layer is the first piezoelectric layer.

5. The ultrasound probe of claim 1,
wherein a front surface of a middle portion in the first piezoelectric layer is recessed more backward than front surfaces of side portions so as to face a back surface of a middle portion in the second piezoelectric layer, and
the back surface of the middle portion in the second piezoelectric layer is protruded more backward than back surfaces of side portions.

6. The ultrasound probe of claim 5,
wherein the first piezoelectric layer and the second piezoelectric layer are protruded and recessed to be engaged with each other with a flexible printed circuit board (FPCB) interposed therebetween.

7. The ultrasound probe of claim 6,
wherein a back surface of the first piezoelectric layer and a front surface of the second piezoelectric layer are formed flat.

8. The ultrasound probe of claim 5,
wherein the middle portion of the first piezoelectric layer is recessed in a stepwise manner with respect to the side portions, and
the middle portion of the second piezoelectric layer is protruded in a stepwise manner with respect to the side portions.

9. The ultrasound probe of claim 1, further comprising:
a backing layer that is located in back of the piezoelectric element unit to block an ultrasound signal propagating to the back of the piezoelectric element unit; and
a lens layer that is located in front of the matching layer to focus an ultrasound signal propagating to the front of the piezoelectric element unit on the target object.
